(19) United States
(12) Patent Application Publication
(10) Pub. No.: US 2019/0244704 A1
(43) Pub. Date: Aug. 8, 2019

(54) DIETARY HABIT MANAGEMENT APPARATUS AND METHOD

(71) Applicant: SAMSUNG ELECTRONICS CO., LTD., Suwon-si (KR)

(72) Inventors: Youn Ho Kim, Hwaseong-si (KR); Byung Hoon Ko, Hwaseong-si (KR); Yunseo Ku, Gwacheon-si (KR); Yong Joo Kwon, Yongin-si (KR); Ui Kun Kwon, Hwaseong-si (KR); Young Soo Kim, Seoul (KR); Jin Young Park, Hwaseong-si (KR); Jong Wook Lee, Suwon-si (KR); Dae Geun Jang, Yongin-si (KR); Chang Mok Choi, Suwon-si (KR); Jae Min Kang, Seoul (KR); Seung Woo Noh, Seongnam-si (KR); Sang Yun Park, Hwaseong-si (KR); Chang Soon Park, Chungju-si (KR); Seung Keun Yoon, Seoul (KR)

(73) Assignee: SAMSUNG ELECTRONICS CO., LTD., Suwon-si (KR)

(21) Appl. No.: 16/266,510

(22) Filed: Feb. 4, 2019

(30) Foreign Application Priority Data
Feb. 5, 2018 (KR) 10-2018-0014202

(51) Int. Cl. G16H 20/60; A61B 5/024; A61B 5/029; A61B 5/0488; A61B 5/145; A61B 5/0402; A61B 5/00

(52) U.S. Cl. CPC G16H 20/60; A61B 5/02416; A61B 5/029; A61B 5/0488; A61B 5/0205; A61B 5/0402; A61B 5/681; A61B 5/0006; A61B 5/14532

(57) ABSTRACT

A dietary habit management apparatus and method are provided. The dietary habit management apparatus includes a bio-signal acquirer configured to acquire a bio-signal of a user, and a processor configured to obtain a total peripheral resistance (TPR) reflected index, from the bio-signal that is acquired by the bio-signal acquirer, and determine whether the user has eaten food, based on the TPR reflected index.

### CROSS-REFERENCE TO RELATED APPLICATION

This application claims priority from Korean Patent Application No. 10-2018-0014202, filed on Feb. 5, 2018, in the Korean Intellectual Property Office, the entire disclosure of which is incorporated herein by reference for all purposes.

### BACKGROUND

### 1. Field

Apparatuses and methods consistent with example embodiments relate to managing dietary habits, based on a bio-signal.

### 2. Description of Related Art

As the food culture has become westernized, the numbers of obese patients and diabetes patients have been increasing, and the importance of controlling food and food portions has been emphasized as a treatment and prevention measure for those patients. In addition, in response to such an interest, diet-friendly restaurants have opened, helping customers' diet and meal portion control.

Most people today get an insufficient amount of exercise because they do not have spare time for exercise, and they often eat large amounts of meals, without a sufficient amount of exercise, which may lead to obesity or diabetes. In addition, with the modern diet including more high-calorie foods than in the past, one is more likely to become diabetic or obese.

### SUMMARY

According to an aspect of an example embodiment, there is provided a dietary habit management apparatus including a bio-signal acquirer configured to acquire a bio-signal of a user, and a processor configured to obtain a total peripheral resistance (TPR) reflected index, from the bio-signal that is acquired by the bio-signal acquirer, and determine whether the user has eaten food, based on the TPR reflected index.

The bio-signal may be one of a pulse pressure signal, a photoplethysmogram (PPG) signal, an electrocardiogram (ECG) signal, an electromyogram (EMG) signal, and a ballistocardiogram (BCG) signal.

The processor may be further configured to extract at least one feature point, from the bio-signal, and obtain the TPR reflected index by combining features corresponding to the at least one feature point.

The TPR reflected index may include any one or any combination of 1/(T₃−T₁), 1/(T₃−T_{sys}), 1/(T₃−Tₘₐₓ), 1/(T₂−T₁), P₂/P₁, P₃/Pₘₐₓ, P₃/P₁, and A_{ppg}/(Pₘₐₓ*A_{dur}), where T₁ denotes a time of a peak point of a first component pulse constituting the bio-signal, T₂ denotes a time of a peak point of a second component pulse constituting the bio-signal, T₃ denotes a time of a peak point of a third component pulse constituting the bio-signal, Tₘₐₓ denotes a time of a peak point of the bio-signal in a first interval, T_{sys} denotes an intermediate time between T₁ and Tₘₐₓ, P₁ denotes an amplitude of the bio-signal at T₁, P₂ denotes an amplitude of the bio-signal at T₂, P₃ denotes an amplitude of the bio-signal at T₃, Pₘₐₓ denotes an amplitude of the bio-signal at Tₘₐₓ, A_{ppg} denotes a sum of amplitudes of the bio-signal of one period, and A_{dur} denotes a sum of amplitudes of the bio-signal in a second interval.

The processor may be further configured to obtain the TPR reflected index, based on a time delay of a plurality of bio-signals that is measured using a plurality of light sources that emits light of different wavelengths.

The processor may be further configured to compare the TPR reflected index or a reciprocal of the TPR reflected index, with a reference value, and determine whether the user has eaten food, based on a result of the TPR reflected index or a reciprocal of the TPR reflected index being compared with the reference value.

The processor may be further configured to set the reference value to be used in determining whether the user has eaten food, based on an instruction of the user or based on the TPR reflected index obtained in a fasting and resting state.

The processor may be further configured to determine a dietary level of the user, based on the TPR reflected index.

The processor may be further configured to acquire exercise data of the user, and correct the TPR reflected index, based on the exercise data.

The processor may be further configured to acquire body temperature data of the user, and correct the TPR reflected index, based on the body temperature.

The processor may be further configured to estimate a blood sugar level of the user, based on the TPR reflected index.

According to an aspect of another example embodiment, there is provided a method of managing dietary habits, the method including acquiring a bio-signal of a user, obtaining a total peripheral resistance (TPR) reflected index, from the bio-signal that is acquired, and determining whether the user has eaten food, based on the TPR reflected index.

The obtaining the TPR reflected index may include extracting at least one feature point, from the bio-signal, and obtaining the TPR reflected index by combining features corresponding to the at least one feature point.

The obtaining the TPR reflected index may include obtaining the TPR reflected index, based on a time delay of a plurality of bio-signals that is measured using a plurality of light sources that emits light of different wavelengths.

The determining of whether the user has eaten food may include comparing the TPR reflected index or a reciprocal of the TPR reflected index, with a reference value, and determining whether the user has eaten food, based on a result of the TPR reflected index or a reciprocal of the TPR reflected index being compared with the reference value.

The method may further include setting the reference value to be used in determining whether the user has eaten food, based on an instruction of the user or based on the TPR reflected index obtained in a fasting and resting state.

The method may further include determining a dietary level of the user, based on the TPR reflected index.

The method may further include acquiring exercise data of the user, and correcting the TPR reflected index, based on the exercise data.

The method may further include acquiring body temperature data of the user, and correcting the TPR reflected index, based on the body temperature data.

The method may further include estimating a blood sugar level of the user, based on the TPR reflected index.

Other features and aspects will be apparent from the following detailed description, the drawings, and the claims.

### BRIEF DESCRIPTION OF THE DRAWINGS

The above and/or other aspects will be more apparent by describing certain example embodiments, with reference to the accompanying drawings, in which:

FIG. 1 is a block diagram illustrating a dietary habit management apparatus according to an example embodiment;

FIG. 2 is a block diagram illustrating a bio-signal acquiring apparatus according to an example embodiment;

FIG. 3 is a block diagram illustrating a processor according to an example embodiment;

FIG. 4 is a graph for describing a TPR reflected index;

FIG. 5 are graphs for describing a method of acquiring Pₙ(P₁, P₂, P₃) and Tₙ(T₁, T₂, T₃) of FIG. 4;

FIG. 6 are graphs for describing a method of acquiring Pₘₐₓ and Tₘₐₓ of FIG. 4;

FIG. 7 are graphs for showing examples of a PPG signal according to food intake;

FIG. 8 are graphs for showing an example of a change in TPR reflected index according to food intake;

FIG. 9 is a block diagram illustrating a processor according to another example embodiment;

FIG. 10 is a graph for describing a relationship between a TPR reflected index and a blood sugar level;

FIG. 11 is a block diagram illustrating a dietary habit management apparatus according to another example embodiment;

FIG. 12 is a flowchart illustrating a method of managing dietary habits according to an example embodiment; and

FIG. 13 is a flowchart illustrating a method of managing dietary habits according to another example embodiment.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those defined matters. Also, well-known functions or constructions are not described in detail because they would obscure the description with unnecessary detail.

In some alternative implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved.

Terms described in below are selected by considering functions in the embodiment and meanings may vary depending on, for example, a user or operator's intentions or customs. Therefore, in the following embodiments, when terms are defined, the meanings of terms may be interpreted based on definitions, and otherwise, may be interpreted based on meanings recognized by those skilled in the art.

As used herein, the singular forms are intended to include the plural forms as well, unless the context clearly indicates otherwise. It will be further understood that the terms “comprises” and/or “comprising,” or “includes” and/or “including” when used in this description, specify the presence of stated features, numbers, steps, operations, elements, components or combinations thereof, but do not preclude the presence or addition of one or more other features, numbers, steps, operations, elements, components or combinations thereof.

It will also be understood that the elements or components in the following description are discriminated in accordance with their respective main functions. In other words, two or more elements may be made into one element or one element may be divided into two or more elements in accordance with a subdivided function. Additionally, each of the elements in the following description may perform a part or whole of the function of another element as well as its main function, and some of the main functions of each of the elements may be performed exclusively by other elements. Each element may be realized in the form of a hardware component, a software component, and/or a combination thereof.

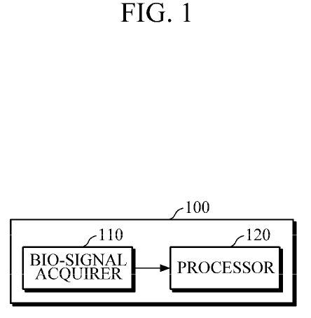

FIG. 1 is a block diagram illustrating a dietary habit management apparatus 100 according to an example embodiment. The dietary habit management apparatus 100 of FIG. 1 may be implemented as a software module or in the form of a hardware chip and may be mounted in an electronic device. In this case, the electronic device may include a mobile phone, a smartphone, a tablet computer, a notebook computer, a personal digital assistant (PDA), a portable multimedia player (PMP), a navigation terminal, an MP3 player, a digital camera, and a wearable device. The wearable device may include wearable devices of a wristwatch type, a wrist band type, a belt type, a necklace type, an ankle band type, a thigh band type, a forearm band type, and the like. However, the electronic device and the wearable device are not limited to the above examples.

Referring to FIG. 1, the dietary habit management apparatus 100 may include a bio-signal acquirer 110 and a processor 120.

The bio-signal acquirer 110 may acquire a bio-signal of a user. Here, the bio-signal may include, but not limited to, a pulse pressure signal, a photoplethysmogram (PPG) signal, an electrocardiogram (ECG) signal, an electromyogram (EMG) signal, a ballistocardiogram (BCG) signal, and the like.

According to one embodiment, the bio-signal acquirer 110 may acquire a bio-signal of the user from an external device. In this case, the bio-signal acquirer 110 may use various communication technologies, such as Bluetooth, Bluetooth low energy (BLE), near field communication (NFC), wireless local area network (WLAN) communication, ZigBee communication, infrared data association (IrDA) communication, Wi-Fi direct (WFD) communication, ultra-wideband (UWB) communication, Ant+ communication, Wi-Fi communication, radio frequency identification (RFID) communication, third generation (3G) communication, fourth generation (4G) communication, fifth generation (5G) communication, and the like.

The external device is a device that measures or stores a bio-signal of a user, and may include, but not limited to, various sensors (e.g., a pulse pressure sensor, a PPG sensor, an ECG sensor, an EMG sensor, a BCG sensor, and the like), a digital TV, a desktop computer, a mobile phone, a smartphone, a tablet computer, a notebook computer, a PDA, a PMP, a navigation terminal, an MP3 player, a digital camera, a wearable device and the like.

According to another embodiment, the bio-signal acquirer 110 may include various sensors for sensing a bio-signal, by which the bio-signal acquirer 110 can directly acquire the user's bio-signal. In this case, the sensor may include a pulse pressure sensor, a PPG sensor, an ECG sensor, an EMG sensor, a BCG sensor, and the like.

The processor 120 may process various signals related to the operations of the dietary habit management apparatus 100.

The processor 120 may control the bio-signal acquirer 110 to acquire the user's bio-signal at predetermined intervals or upon request of the user and may obtain or extract a total peripheral resistance (TPR) reflected index (hereinafter will be referred to as a “TPR reflected index”) from the acquired bio-signal. In this case, the TPR reflected index may be an index in negative correlation or positive correlation with TPR. For example, the processor 120 may extract a TPR reflected index by extracting feature points from the bio-signal and combining features corresponding to the extracted feature points, or extract a TPR reflected index using a time delay between a plurality of bio-signals measured using a plurality of light sources that emit light of different wavelengths. In this case, the time delay is a time difference between the bio-signals and is obtained by extracting feature points that respectively correspond to the plurality of bio-signals and calculating the time difference between the extracted feature points.

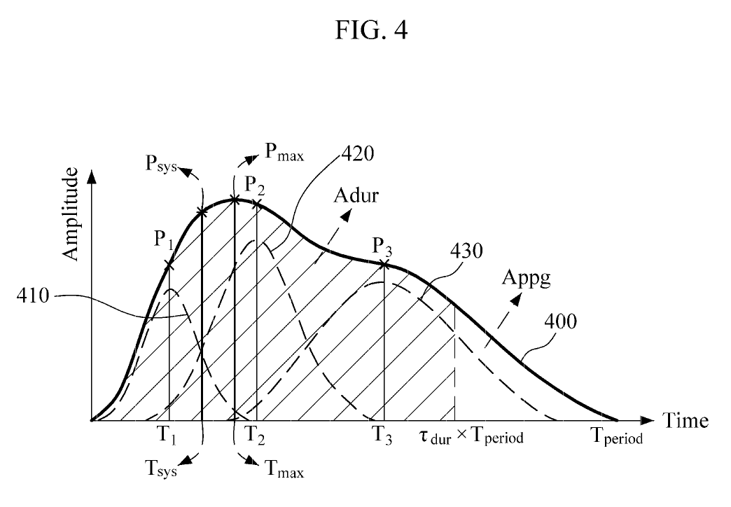

The TPR reflected index will be described below in detail with reference to FIG. 4.

In addition, the processor 120 may determine whether the user has eaten food by analyzing the extracted TPR reflected index. For example, the processor 120 may compare the TPR reflected index (when the TPR reflected index is in negative correlation with TPR) or the reciprocal of the TPR reflected index (when the TPR reflected index is in positive correlation with TPR) with a predetermined reference value and determine that the user has eaten food when the TPR reflected index or the reciprocal of the TPR reflected index is greater than the predetermined reference value.

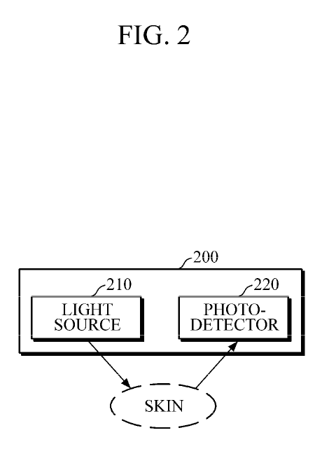

FIG. 2 is a block diagram illustrating a bio-signal acquiring apparatus 200 according to an example embodiment. The bio-signal acquiring apparatus 200 of FIG. 2 may be one embodiment of the bio-signal acquirer 110 of FIG. 1.

Referring to FIG. 2, the bio-signal acquiring apparatus 200 may include a light source 210 and a photodetector 220.

The light source 210 may emit light to the skin of a user. The light source 210 may include at least one light source formed by a light emitting diode (LED), a laser diode, or a phosphor.

According to one embodiment, each of the light sources may emit visible ray light, near infrared ray (NIR) light, or mid-infrared ray (MIR) light. However, the wavelength of the light emitted from each of the light sources may vary depending on the purpose of measurement or a target component to be analyzed. In addition, each of the light sources is not necessarily configured with a single light emitting structure, and may be formed as an array composed of a plurality of light emitting structures. In this case, each of the light sources may emit light of the same wavelengths or emit light of a different wavelength.

The light source 210 may further include various optical devices to allow light to be emitted to a desired position.

The photodetector 220 may receive light reflected or scattered from the skin of the user and acquire the user's bio-signal (e.g., PPG signal). The photodetector 220 may include one or more photodetectors formed by a photodiode, a photo transistor (PTr), or a charge-coupled device (CCD). The photodetector is not necessarily configured with a single device and may be formed as an array composed of a plurality of devices.

The numbers and arrangements of the light sources and the photodetectors may vary depending on the purpose of use of the bio-signal acquiring apparatus 200 and the size and the shape of the electronic device in which the bio-signal acquiring apparatus 200 is mounted.

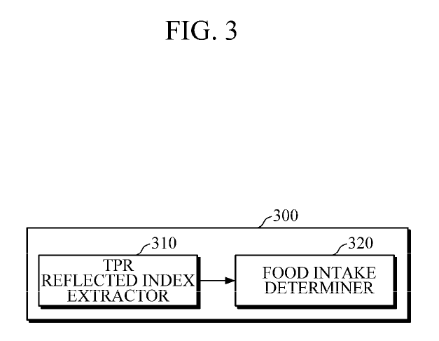

FIG. 3 is a block diagram illustrating a processor 300 according to an example embodiment. The processor 300 of FIG. 3 may be one embodiment of the processor 120 of FIG. 1.

Referring to FIG. 3, the processor 300 may include a TPR reflected index extractor 310 and a food intake determiner 320 for determining whether a user has eaten food.

The TPR reflected index extractor 310 may extract a TPR reflected index from a bio-signal.

According to one embodiment, the TPR reflected index extractor 310 may extract one or more feature points by analyzing the bio-signal and extract a TPR reflected index by combining features corresponding to the one or more extracted feature points. In this case, the feature points may include a peak point of the bio-signal and an intermediate point of a peak point of each component pulse constituting the bio-signal and the peak point of the bio-signal, but these are an embodiment and aspects of the disclosure are not limited thereto.

According to another embodiment, the TPR reflected index extractor 310 may calculate a time delay between a plurality of bio-signals by analyzing the plurality of bio-signal, which are measured using a plurality of light sources that emit light of different wavelengths and extract a TPR reflected index on the basis of the calculated time delay. For example, the TPR reflected index extractor 310 may extract feature points corresponding to each other from the plurality of bio-signals and calculate a time delay between the plurality of bio-signals by computing a time difference between the extracted feature points. In addition, the TPR reflected index extractor 310 may extract the TPR reflected index using Equation 1 below.

TPR reflected index=a*T−b*k (1)

Here, T denotes a time delay between a first bio-signal and a second bio-signal having a wavelength different from that of the first bio-signal, k denotes a heart-rate reflected index or a cardiac-output reflected index, and a and b may be scale constants. In this case, k may be obtained by analyzing the first bio-signal and/or the second bio-signal, or may be obtained by acquiring and analyzing another bio-signal. For example, the cardiac-output reflected index may be obtained by extracting one or more feature points from a bio-signal (the first bio-signal, the second bio-signal, or another bio-signal) and combining features (e.g., Pₘₐₓ/Pₐᵣₑₐ, Pₘₐₓ/P₃, P_{sys}/P₃, P₁/P₃, P₂/P₃, and 1/T_{period}, refer to FIG. 4) corresponding to the one or more extracted feature points, and the heart-rate reflected index may be obtained by dividing the cardiac-output reflected index by a stroke volume.

The food intake determiner 320 may determine whether the user has eaten food by analyzing the TPR reflected index. According to one embodiment, the food intake determiner 320 may compare the TPR reflected index (when the TPR reflected index is in negative correlation with TPR) or the reciprocal of the TPR reflected index (when the TPR reflected index is in positive correlation with TPR) with a predetermined reference value and determine that the user has eaten food when the TPR reflected index or the reciprocal of the TPR reflected index is greater than the predetermined reference value.

The food intake determiner 320 may determine a dietary level of the user by analyzing the TPR reflected index. For example, the dietary level may be classified into a plurality of levels (e.g., a first level, a second level, and a third level) according to a value of the TPR reflected index (when the TPR reflected index is in negative correlation with TPR) or the reciprocal of the TPR reflected index (when the TPR reflected index is in positive correlation with TPR). In this case, the food intake determiner 320 may determine a level at which the user's TPR reflected index (when the TPR reflected index is in negative correlation with TPR) or the reciprocal of the TPR reflected index of the user (when the TPR reflected index is in positive correlation with TPR) is situated, and determine the user's dietary level according to the determined level. In this case, when the level is higher (e.g., the first level<the second level<the third level), the food intake determiner 320 may determine that the user has eaten higher-calorie food or higher glycemic index food.

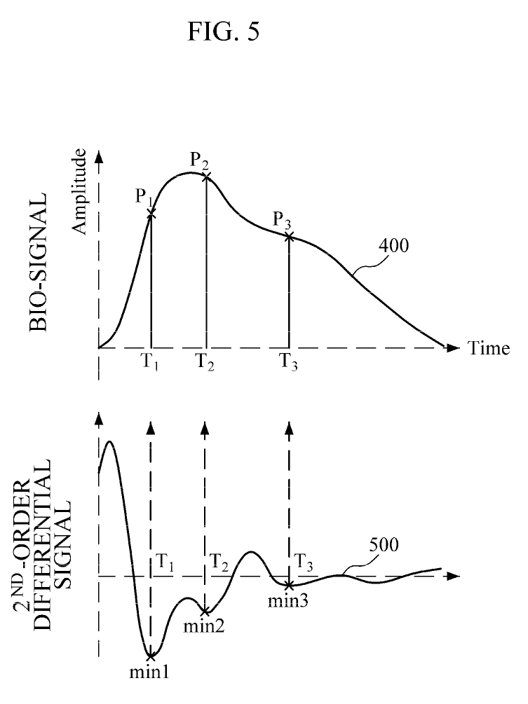

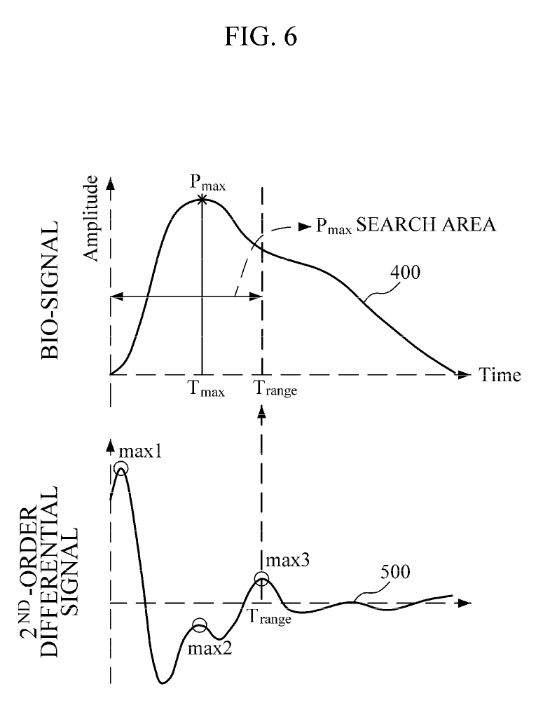

FIG. 4 is a graph for describing a TPR reflected index, FIG. 5 are graphs for describing a method of acquiring Pₙ(P₁, P₂, P₃) and Tₙ(T₁, T₂, T₃) of FIG. 4, and FIG. 6 are graphs for describing a method of acquiring Pₘₐₓ and Tₘₐₓ of FIG. 4. In this case, it is assumed that a bio-signal is a PPG signal and the TPR reflected index is in positive correlation with TPR.

Referring to FIG. 4, a waveform of a PPG signal 400 may be a summation of a propagation wave 410 propagating from the heart to peripheral parts of a body and reflection waves 420 and 430 returning from the peripheral parts of the body. That is, the PPG signal 400 may be a summation of three or more component pulses 410 to 430. In this case, reference numeral 400 denotes the PPG signal of one period T_{period}, 410 denotes a first component pulse, 420 denotes a second component pulse, and 430 denotes a third component pulse. In addition, T₁ denotes the time of the peak point of the first component pulse 410, P₁ denotes the amplitude of the PPG signal 400 at T₁, T₂ denotes the time of the peak point of the second component pulse 420, P₂ denotes the amplitude of the PPG signal 400 at T₂, T₃ denotes the time of the peak point of the third component pulse 430, P₃ denotes the amplitude of the PPG signal 400 at T₃, Tₘₐₓ denotes the time of the peak point of the PPG signal 400 in a predetermined interval, Pₘₐₓ denotes the amplitude of the PPG signal 400 at Tₘₐₓ, T_{sys} denotes the intermediate time between T₁ and Tₘₐₓ P_{sys} denotes the amplitude of the PPG signal 400 at T_{sys}, τ_{dur} denotes a setting factor (0≤τ_{dur}≤1) (e.g., 0.7) of the system, A_{dur} denotes the sum of amplitudes of the PPG signal 400 between time 0 and t_{dur}*T_{period}, and A_{ppg} denotes the sum of amplitudes of the PPG signal of one period T_{period}.

Within the PPG signal 400, as T₃ or T₂ increases, the TPR reflected index may decrease, and as T₁, T_{sys}, or Tₘₐₓ increases, the TPR reflected index may increase. In addition, within the PPG signal 400, as P₂, P₃, or A_{ppg} increases, the TPR reflected index may increase, and as P₁ or Pₘₐₓ increases, the TPR reflected index may decrease. For example, the TPR reflected index may include 1/(T₃−T₁), 1/(T₃−T_{sys}), 1/(T₃−Tₘₐₓ), 1/(T₂−T₁), P₂/P₁, P₃/Pₘₐₓ, P₃/P₁, A_{ppg}/(Pₘₐₓ*A_{dur}), and the like.

Although it is described in FIG. 4 that T_{sys} is the intermediate time between T₁ and Tₘₐₓ the disclosure is not limited thereto. That is, T_{sys} may be an arbitrary internally dividing point between T₁ and Tₘₐₓ or an arbitrary internally dividing point between T₁ and T₂.

Referring to FIG. 5, Pₙ(P₁, P₂, P₃), and Tₙ(T₁, T₂, T₃) of FIG. 4 may be obtained based on a second-order differential signal 500 of the PPG signal 400. When the second-order differential signal 500 is obtained from the PPG signal 400, the second-order differential signal 500 includes a plurality of local minimum points min1, min2, and min3. When the local minimum points min1 to min3 included in the second-order differential signal 500 are arranged in a time-order sequence, the local minimum point min1 corresponds to T₁, the local minimum point min2 corresponds to T₂, and the local minimum point min3 corresponds to T₃. In addition, the amplitude of the PPG signal 400 at T₁ corresponds to P₁, the amplitude of the PPG signal 400 at T₂ corresponds to P₂, and the amplitude of the PPG signal 400 at T₃ corresponds to P₃.

Referring to FIG. 6, Pₘₐₓ and Tₘₐₓ of FIG. 4 may be obtained based on the second-order differential signal 500 of the PPG signal 400. When the second-order differential signal 500 is obtained from the PPG signal 400, the second-order differential signal 500 includes a plurality of local maximum points max1, max2, and max3. When the local maximum points max1 to max3 included in the second-order differential signal 500 are arranged in a time-order sequence and the time corresponding to the third maximum point max3 is T_{range}, the time of the peak point of the PPG signal 400 in the range of 0≤time≤T_{range} corresponds to Tₘₐₓ and the amplitude of the PPG signal 400 at Tₘₐₓ corresponds to Pₘₐₓ.

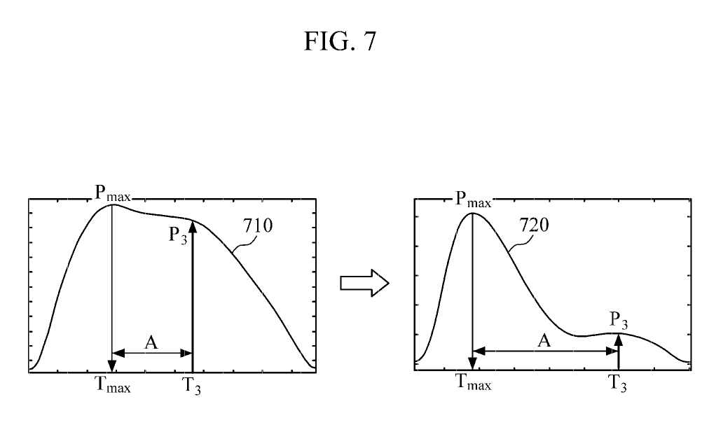

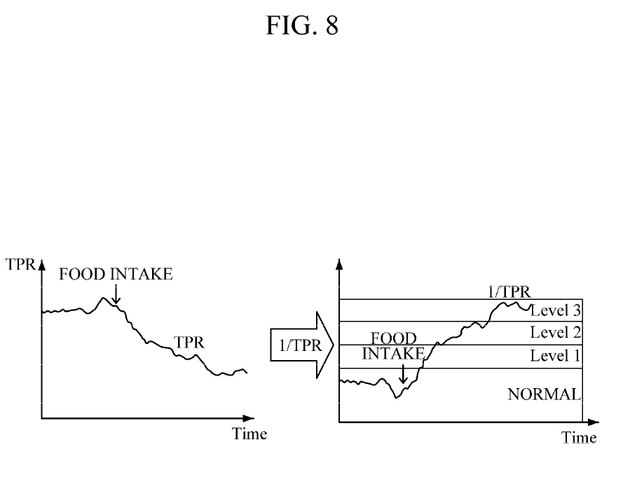

FIG. 7 are graphs for showing examples of a PPG signal according to food intake, and FIG. 8 are graphs for showing an example of a change in TPR reflected index according to food intake.

As shown in FIG. 7, when a PPG signal 710 before food intake is compared with a PPG signal 720 after alcohol consumption, P3 decreases after food intake, and also A value (T₃−Tₘₐₓ) increases. That is, the A value (T₃−Tₘₐₓ) increases due to food intake, and as shown in FIG. 8, the TPR reflected index (e.g., 1/(T₃−Tₘₐₓ)) decreases according to the food intake. This may be interpreted that the diameter of peripheral blood vessels increases and the blood flow increases.

Therefore, a dietary habit management apparatus (e.g., 100 in FIG. 1) may monitor the TPR reflected index (e.g., 1/(T₃−Tₘₐₓ)) and determine that the user is eating food (alcohol) when the reciprocal of the TPR reflected index exceeds a predetermined reference value. In addition, when a dietary level is classified into a first level, a second level, and a third level, the dietary habit management apparatus (e.g., 100 in FIG. 1) may determine the user's dietary level by identifying a level at which the reciprocal of the TPR reflected index is situated. In this case, the dietary habit management apparatus (e.g., 100 in FIG. 1) may determine that the user has eaten higher calorie food or higher glycemic index food when the level is higher (e.g., first level<second level<third level). That is, as the level is increased from level 1 to level 3, it may be determined that the user has eaten higher calorie food or higher glycemic index food, and thereby it is possible to manage blood sugar level and calories, as well as dietary habits through storage of the number of meals and times of meals.

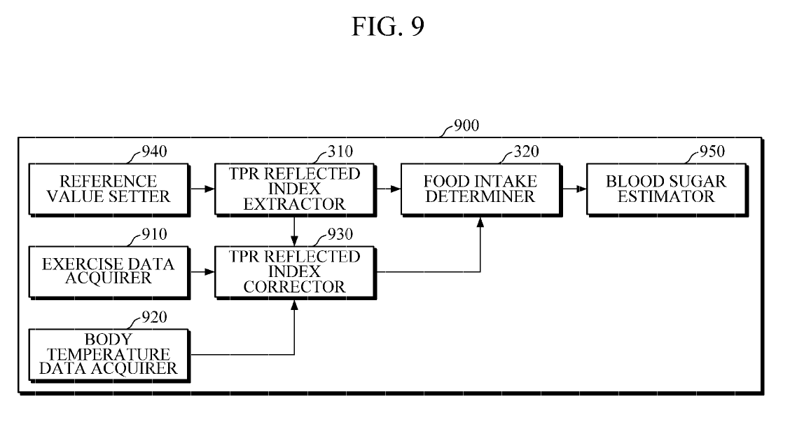

FIG. 9 is a block diagram illustrating a processor 900 according to another example embodiment. The processor 900 of FIG. 9 may be one embodiment of the processor 120 of FIG. 1.

Referring to FIG. 9, the processor 900 may include an exercise data acquirer 910, a body temperature data acquirer 920, a TPR reflected index extractor 310, a TPR reflected index corrector 930, a reference value setter 940, a food intake determiner 320, and a blood sugar estimator 950. Here, the TPR reflected index extractor 310 and the food intake determiner 320 are the same as those described with reference to FIG. 3, and hence detailed descriptions thereof will not be reiterated.

The exercise data acquirer 910 may acquire exercise data of a user.

According to one embodiment, the exercise data acquirer 910 may receive and acquire the user's exercise data from an external device. In this case, the exercise data acquirer 910 may use various communication technologies, such as Bluetooth, BLE, NFC, WLAN communication, ZigBee communication, IrDA communication, WFD communication, UWB communication, Ant+ communication, Wi-Fi communication, RFID communication, 3G communication, 4G communication, 5G communication, and the like.

The external device is a device that measures or stores user's exercise data and may include, but not limited to, various sensors (e.g., accelerator sensor, a gyro sensor, and the like), a digital TV, a desktop computer, a mobile phone, a smartphone, a tablet computer, a notebook computer, a PDA, a PMP, a navigation terminal, an MP3 player, a digital camera, a wearable device, and the like.

According to another embodiment, the exercise data acquirer 910 may include various sensors that sense the user's exercise data and directly obtain the user's exercise data through the various sensors. In this case, the sensors may include, but not limited to, an acceleration sensor, a gyro sensor, and the like.

The body temperature data acquirer 920 may acquire body temperature data of the user.

According to one embodiment, the body temperature data acquirer 920 may receive and acquire the user's body temperature data from an external device. In this case, the body temperature data acquirer 920 may use various communication technologies, such as Bluetooth, BLE, NFC, WLAN communication, ZigBee communication, IrDA communication, WFD communication, UWB communication, Ant+ communication, Wi-Fi communication, RFID communication, 3G communication, 4G communication, 5G communication, and the like.

The external device may be a device that measures or stores the user's body temperature data and may include, but not limited to, a temperature sensor, a digital TV, a desktop computer, a mobile phone, a smartphone, a tablet computer, a notebook computer, a PDA, a PMP, a navigation terminal, an MP3 player, a digital camera, a wearable device, and the like.

According to another embodiment, the body temperature data acquirer 920 may include a temperature sensor that senses the user's body temperature and may directly acquire the user's body temperature data using the temperature sensor.

The TPR reflected index corrector 930 may correct a TPR reflected index based on the user's exercise data and/or body temperature data.

The TPR reflected index is related to the expansion of the blood vessels, and hence may be affected not only by food intake but also by other factors, such as exercise intensity, body temperature, and the like.

According to one embodiment, the TPR reflected index corrector 930 may determine the amount of exercise of the user on the basis of the user's exercise data and increase or decrease the TPR reflected index according to the amount of exercise of the user. In this case, the specified increase or decrease amount of TPR reflected index may be determined using an exercise amount-TPR model that defines a relationship between the amount of exercise of the user and the TPR reflected index.

According to another embodiment, the TPR reflected index corrector 930 may increase or decrease the TPR reflected index according to the user's body temperature. In this case, the specified increase or decrease amount of TPR reflected index may be determined using a body temperature-TPR model that defines a relationship between the user's body temperature and the TPR reflected index.

The exercise amount-TPR model and the body temperature-TPR model may be constructed in advance using regression analysis or machine learning and be stored in the processor 900 or in an external database.

The reference value setter 940 may set a reference value to be used in determining whether the user has eaten food. For example, the reference value setter 940 may set the reference value according to a user's instruction or on the basis of the TPR reflected index extracted in a fasting and resting state. Here, the resting state may refer to a state in which the user is motionless or a state in which the user's exercise intensity is less than or equal to a predetermined threshold value.

The blood sugar estimator 950 may estimate a user's blood sugar level based on the TPR reflected index. For example, the blood sugar estimator 950 may estimate the user's blood sugar level using a TPR-blood sugar model that defines a relationship between the TPR reflected index and the blood sugar. In this case, the TPR-blood sugar model may be constructed in advance using regression analysis or machine learning and be stored in the processor 900 or in an external database.

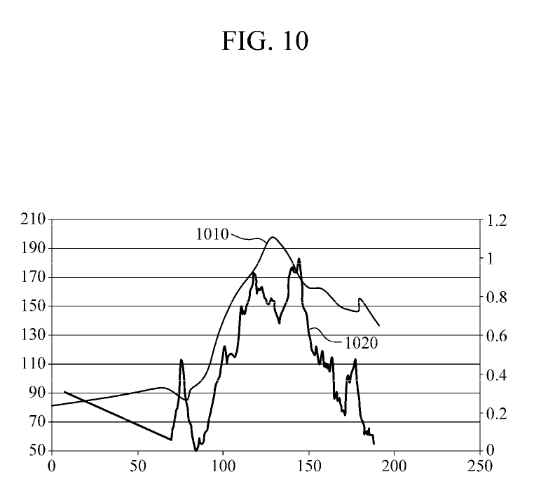

FIG. 10 is a graph for describing a relationship between a TPR reflected index and a blood sugar level. FIG. 10 is a graph showing a blood sugar level measurement result and a change in the TPR reflected index extracted from a PPT signal.

In the illustrated example, a blood sugar level 1010 shows a tendency to increase from the start of the meal (about 64 minutes) until 120 minutes and decrease since then. A reciprocal 1020 of the TPR reflected index (when the TPR reflected index is in positive correlation with TPR) also shows a tendency to increase and then decrease in a similar pattern as the blood sugar level 1010.

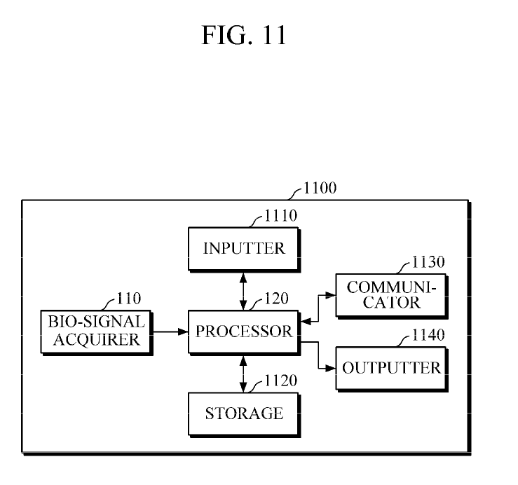

FIG. 11 is a block diagram illustrating a dietary habit management apparatus 1100 according to another example embodiment. The dietary habit management apparatus of FIG. 11 may be implemented as a software module or in the form of a hardware chip and may be mounted in an electronic device. The electronic device may include, but not limited to, a mobile phone, a smartphone, a tablet computer, a notebook computer, a PDA, a PMP, a navigation terminal, an MP3 player, a digital camera, a wearable device and the like. The wearable device may include wearable devices of a wristwatch type, a wrist band type, a belt type, a necklace type, an ankle band type, a thigh band type, a forearm band type, and the like. However, the electronic device and the wearable device are not limited to the above examples.

Referring to FIG. 11, the dietary habit management apparatus 1100 may include a bio-signal acquirer 110, a processor 120, an inputter 1110, a storage 1120, a communicator 1130, and an outputter 1140. Here, the bio-signal acquirer 110 and the processor 120 are the same those described with reference to FIGS. 1 to 10, and thus detailed descriptions thereof will not be reiterated.

The inputter 1110 may receive various operation signals input by a user. According to one embodiment, the inputter 1110 may include a key pad, a dome switch, a resistive or capacitive touch pad, a jog wheel, a jog switch, a hardware (H/W) button, and the like. When a touch pad has a layered structure with a display, this structure may be referred to as a touch screen.

Programs or instructions for operations of the dietary habit management apparatus 1100 may be stored in the storage 1120 and data input to and output from the dietary habit management apparatus 1100 may also be stored in the storage 1120. In addition, the storage 1120 may store bio-signal data acquired through the bio-signal acquirer 110, TPR reflected index data extracted by the processor 120, data about whether the user has eaten food and the dietary level, which is determined by the processor 120, blood sugar level data of the user estimated by the processor 120, and various models (e.g., an exercise-TPR model, a body temperature-TPR model, a TPR-blood sugar model, etc.).

The storage 1120 may include at least one type of storage media, such as a flash memory, a hard disk type memory, a multimedia card micro type memory, a card-type memory (e.g., SD or XD memory), random access memory (RAM), static random access memory (SRAM), read only memory (ROM), electrically erasable programmable read only memory (EEPROM), programmable read only memory (PROM), magnetic memory, and optical disk. In addition, the dietary habit management apparatus 1100 may operate an external storage medium, such as web storage providing a storage function of the storage 1120.

The communicator 1130 may communicate with an external device. For example, the communicator 1130 may transmit the bio-signal data acquired through the bio-signal acquirer 110, the TPR reflected index data extracted by the processor 120, the data about whether the user has eaten food and the dietary level that is determined by the processor 120, the blood sugar level data of the user estimated by the processor 120, and various models (e.g., an exercise-TPR model, a body temperature-TPR model, a TPR-blood sugar model, etc.) to the external device, or receive a variety of data helpful to determine the food intake of the user and the dietary level and estimate the user's blood sugar level from the external device.

Here, the external device may be medical equipment that uses the data input by the user through the inputter 1110, the bio-signal data acquired through the bio-signal acquirer 110, the TPR reflected index data extracted by the processor 120, the data about whether the user has eaten food and the dietary level that is determined by the processor 120, the blood sugar level data of the user estimated by the processor 120, and various models (e.g., an exercise-TPR model, a body temperature-TPR model, a TPR-blood sugar model, etc.), or a printer or display device to output a result. In addition, the external device may include, but not limited to, a digital TV, a desktop computer, a mobile phone, a smartphone, a tablet computer, a notebook computer, a PDA, a PMP, a navigation terminal, an MP3 player, a digital camera, a wearable device, and the like.

The communicator 1130 may communicate with the external device using various communication technologies, such as Bluetooth, BLE, NFC, WLAN communication, ZigBee communication, IrDA communication, WFD communication, UWB communication, Ant+ communication, Wi-Fi communication, RFID communication, 3G communication, 4G communication, 5G communication, and the like. However, these are examples, and aspects of the disclosure are not limited thereto.

The outputter 1140 may output the data input by the user through the inputter 1110, the bio-signal data acquired through the bio-signal acquirer 110, the TPR reflected index data extracted by the processor 120, the data about whether the user has eaten food and the dietary level that is determined by the processor 120, the blood sugar level data of the user estimated by the processor 120, and the like. According to one embodiment, the outputter 1140 may output the data input by the user through the inputter 1110, the bio-signal data acquired through the bio-signal acquirer 110, the TPR reflected index data extracted by the processor 120, the data about whether the user has eaten food and the dietary level that is determined by the processor 120, the blood sugar level data of the user estimated by the processor 120, and the like in any one or any combination of visual, audible, and tactile manners. To this end, the outputter 1140 may include a display, a speaker, a vibrator, and the like.

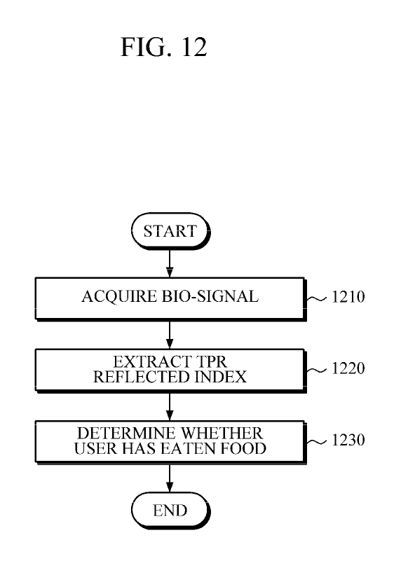

FIG. 12 is a flowchart illustrating a method of managing dietary habits according to an example embodiment. The method shown in FIG. 12 may be performed by the dietary habit management apparatus 100 of FIG. 1.

Referring to FIGS. 1 and 12, the dietary habit management apparatus 100 may acquire a user's bio-signal in operation 1210. Here, the bio-signal may include, but not limited to, a pulse pressure signal, a PPG signal, an ECG signal, an EMG signal, a BCG signal, and the like.

For example, the dietary habit management apparatus 100 may acquire the user's bio-signal from an external device that measures or stores the user's bio-signal, or may include various sensors that sense the bio-signal and obtain the user's bio-signal through the various sensors.

The dietary habit management apparatus 100 may extract a TPR reflected index from the bio-signal in operation 1220.

According to one embodiment, the dietary habit management apparatus 100 may extract one or more feature points by analyzing the bio-signal and extract the TPR reflected index by combining features corresponding to the one or more extracted feature points.

According to another embodiment, the dietary habit management apparatus 100 may calculate a time delay between a plurality of bio-signals by analyzing the plurality of bio-signal that are measured using a plurality of light sources that emit light of different wavelengths, and may extract a TPR reflected index on the basis of the calculated time delay. For example, the TPR reflected index extractor 310 may extract the TPR reflected index using Equation 1.

The dietary habit management apparatus 100 may determine whether the user has eaten food by analyzing the TPR reflected index in operation 1230. According to one embodiment, the dietary habit management apparatus 100 may compare the TPR reflected index (when the TPR reflected index is in negative correlation with TPR) or the reciprocal of the TPR reflected index (when the TPR reflected index is in positive correlation with TPR) with a predetermined reference value and determine that the user has eaten food when the TPR reflected index or the reciprocal of the TPR reflected index is greater than the predetermined reference value.

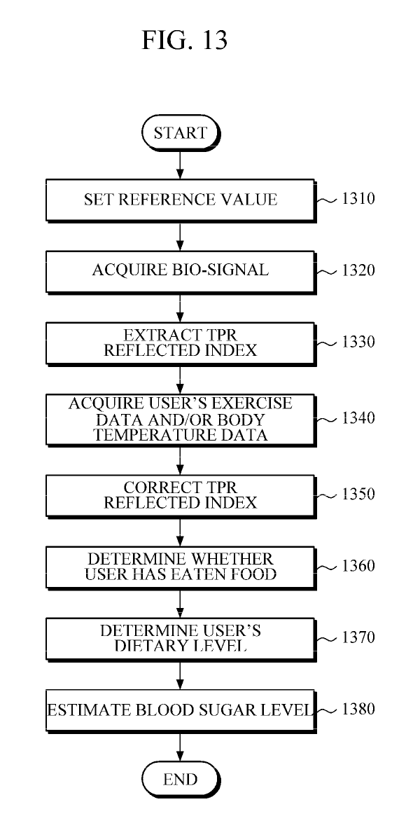

FIG. 13 is a flowchart illustrating a method of managing dietary habits according to another example embodiment. The method shown in FIG. 13 may be performed by the dietary habit management apparatus 100 of FIG. 1.

Referring to FIGS. 1 and 13, the dietary habit management apparatus 100 may set a reference value to be used to determine whether the user has eaten food in operation 1310. For example, the dietary habit management apparatus 10 may set the reference value according to a user's instruction or on the basis of the TPR reflected index extracted in a fasting and resting state. Here, the resting state may refer to a state in which the user is motionless or a state in which the user's exercise intensity is less than or equal to a predetermined threshold value.

The dietary habit management apparatus 100 may acquire a user's bio-signal in operation 1320. For example, the dietary habit management apparatus 100 may acquire the user's bio-signal from an external device that measures or stores the user's bio-signal, or may include various sensors that sense a bio-signal and directly acquire the user's bio-signal through the various sensors.

The dietary habit management apparatus 100 may extract a TPR reflected index from the bio-signal in operation 1330.

According to one embodiment, the dietary habit management apparatus 100 may extract one or more feature points by analyzing the bio-signal and extract the TPR reflected index by combining features corresponding to the one or more extracted feature points.

According to another embodiment, the dietary habit management apparatus 100 may calculate a time delay between a plurality of bio-signals by analyzing the plurality of bio-signal that are measured using a plurality of light sources that emit light of different wavelengths, and may extract a TPR reflected index on the basis of the calculated time delay. For example, the TPR reflected index extractor 310 may extract the TPR reflected index using Equation 1.

The dietary habit management apparatus 100 may acquire the user's exercise data and/or body temperature data in operation 1340. For example, the dietary habit management apparatus 100 may acquire the user's exercise data and/or body temperature data from an external device that measures or stores the user's exercise data and/or body temperature data, or may include various sensors that sense the user's exercise data and/or body temperature data and directly acquire the user's exercise data and/or body temperature data through the various sensors.

The dietary habit management apparatus 100 may correct the TPR reflected index based on the user's exercise data and/or body temperature data in operation 1350. For example, the dietary habit management apparatus 100 may determine the amount of exercise of the user on the basis of the user's exercise data, increase or decrease the TPR reflected index according to the amount of exercise of the user, or increase or decrease the TPR reflected index according to the body temperature of the user. In this case, the dietary habit management apparatus 100 may use an exercise amount-TPR model and/or a body temperature-TPR model.

The dietary habit management apparatus 100 may determine whether the user has eaten food by analyzing the TPR reflected index in operation 1360. According to one embodiment, the dietary habit management apparatus 100 may compare the TPR reflected index (when the TPR reflected index is in negative correlation with TPR) or the reciprocal of the TPR reflected index (when the TPR reflected index is in positive correlation with TPR) with a predetermined reference value and determine that the user has eaten food when the TPR reflected index or the reciprocal of the TPR reflected index is greater than the predetermined reference value.

The dietary habit management apparatus 100 may determine a dietary level of the user by analyzing the TPR reflected index in operation 1370. For example, the dietary level may be classified into a plurality of levels (e.g., a first level, a second level, and a third level) according to a value of the TPR reflected index (when the TPR reflected index is in negative correlation with TPR) or the reciprocal of the TPR reflected index (when the TPR reflected index is in positive correlation with TPR). In this case, the dietary habit management apparatus 100 may determine a level at which the user's TPR reflected index (when the TPR reflected index is in negative correlation with TPR) or the reciprocal of the TPR reflected index of the user (when the TPR reflected index is in positive correlation with TPR) is situated, and determine the user's dietary level according to the determined level. In this case, when the level is higher (e.g., the first level<the second level<the third level), the food intake determiner 320 may determine that the user has eaten higher-calorie food or higher glycemic index food.

The dietary habit management apparatus 100 may estimate a user's blood sugar level based on the TPR reflected index in operation 1380. For example, the dietary habit management apparatus 100 may estimate the user's blood sugar level using the TPR-blood sugar model.

The current embodiments can be implemented as computer readable codes in a computer readable record medium. Codes and code segments constituting the computer program can be easily inferred by a skilled computer programmer in the art. The computer readable record medium includes all types of record media in which computer readable data are stored. Examples of the computer readable record medium include a ROM, a RAM, a CD-ROM, a magnetic tape, a floppy disk, and an optical data storage. Further, the record medium may be implemented in the form of a carrier wave such as Internet transmission. In addition, the computer readable record medium may be distributed to computer systems over a network, in which computer readable codes may be stored and executed in a distributed manner.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A dietary habit management apparatus comprising:
a bio-signal acquirer configured to acquire a bio-signal of a user; and
a processor configured to:
obtain a total peripheral resistance (TPR) reflected index, from the bio-signal acquired by the bio-signal acquirer; and
determine whether the user has eaten food, based on the TPR reflected index.

2. The dietary habit management apparatus of claim 1, wherein the bio-signal is one of a pulse pressure signal, a photoplethysmogram (PPG) signal, an electrocardiogram (ECG) signal, an electromyogram (EMG) signal, and a ballistocardiogram (BCG) signal.

3. The dietary habit management apparatus of claim 1, wherein the processor is further configured to:
extract at least one feature point, from the bio-signal; and
obtain the TPR reflected index by combining features corresponding to the at least one feature point.

4. The dietary habit management apparatus of claim 3, wherein the TPR reflected index comprises any one or any combination of 1/(T₃−T₁), 1/(T₃−T_{sys}), 1/(T₃−Tₘₐₓ), 1/(T₂−T₁), P₂/P₁, P₃/Pₘₐₓ, P₃/P₁, and A_{ppg}/(Pₘₐₓ*A_{dur}), where T₁ denotes a time of a peak point of a first component pulse constituting the bio-signal, T₂ denotes a time of a peak point of a second component pulse constituting the bio-signal, T₃ denotes a time of a peak point of a third component pulse constituting the bio-signal, Tₘₐₓ denotes a time of a peak point of the bio-signal in a first interval, T_{sys} denotes an intermediate time between T₁ and Tₘₐₓ, P₁ denotes an amplitude of the bio-signal at T₁, P₂ denotes an amplitude of the bio-signal at T₂, P₃ denotes an amplitude of the bio-signal at T₃, Pₘₐₓ denotes an amplitude of the bio-signal at Tₘₐₓ, A_{ppg} denotes a sum of amplitudes of the bio-signal of one period, and A_{dur} denotes a sum of amplitudes of the bio-signal in a second interval.

5. The dietary habit management apparatus of claim 1, wherein the processor is further configured to obtain the TPR reflected index, based on a time delay of a plurality of bio-signals that is measured using a plurality of light sources that emits light of different wavelengths.

6. The dietary habit management apparatus of claim 1, wherein the processor is further configured to:
compare the TPR reflected index or a reciprocal of the TPR reflected index with a reference value; and
determine whether the user has eaten food, based on a result of the TPR reflected index or a reciprocal of the TPR reflected index being compared with the reference value.

7. The dietary habit management apparatus of claim 6, wherein the processor is further configured to set the reference value to be used in determining whether the user has eaten food, based on an instruction of the user or based on the TPR reflected index obtained in a fasting and resting state.

8. The dietary habit management apparatus of claim 1, wherein the processor is further configured to determine a dietary level of the user, based on the TPR reflected index.

9. The dietary habit management apparatus of claim 1, wherein the processor is further configured to:
acquire exercise data of the user; and
correct the TPR reflected index, based on the exercise data.

10. The dietary habit management apparatus of claim 1, wherein the processor is further configured to:
acquire body temperature data of the user; and
correct the TPR reflected index, based on the body temperature data.

11. The dietary habit management apparatus of claim 1, wherein the processor is further configured to estimate a blood sugar level of the user, based on the TPR reflected index.

12. A method of managing dietary habits, the method comprising:
acquiring a bio-signal of a user;
obtaining a total peripheral resistance (TPR) reflected index, from the bio-signal; and
determining whether the user has eaten food, based on the TPR reflected index.

13. The method of claim 12, wherein the obtaining the TPR reflected index comprises:
extracting at least one feature point, from the bio-signal and
obtaining the TPR reflected index by combining features corresponding to the at least one feature point.

14. The method of claim 12, wherein the obtaining the TPR reflected index comprises obtaining the TPR reflected index, based on a time delay of a plurality of bio-signals that is measured using a plurality of light sources that emits light of different wavelengths.

15. The method of claim 12, wherein the determining whether the user has eaten food comprises:
comparing the TPR reflected index or a reciprocal of the TPR reflected index with a reference value; and
determining whether the user has eaten food, based on a result of the TPR reflected index or a reciprocal of the TPR reflected index being compared with the reference value.

16. The method of claim 15, further comprising setting the reference value to be used in determining whether the user has eaten food, based on an instruction of the user or based on the TPR reflected index obtained in a fasting and resting state.

17. The method of claim 12, further comprising determining a dietary level of the user, based on the TPR reflected index.

18. The method of claim 12, further comprising:
acquiring exercise data of the user; and
correcting the TPR reflected index based on the exercise data.

19. The method of claim 12, further comprising:
acquiring body temperature data of the user; and
correcting the TPR reflected index based on the body temperature data.

20. The method of claim 12, further comprising estimating a blood sugar level of the user, based on the TPR reflected index.
